(19) United States
(12) Patent Application Publication
(10) Pub. No.: US 2013/0226286 A1
(43) Pub. Date: Aug. 29, 2013

(54) PROSTHETIC AORTIC CONDUIT WITH REPLACEMENT VALVE LOCATING MEANS

(71) Applicant: Jonathon David Hargreaves, Kilmalcolm Renfrewshire Strathclyde (GB)

(72) Inventor: Jonathon David Hargreaves, Kilmalcolm Renfrewshire Strathclyde (GB)

(73) Assignee: Vascutek Limited, Renfrewshire (GB)

(21) Appl. No.: 13/883,303

(22) PCT Filed: Nov. 15, 2011
(86) PCT No.: PCT/GB2011/052229

§ 371 Date: May 3, 2013

(30) Foreign Application Priority Data
Nov. 16, 2010 (GB) 1019354.8

(51) Int. Cl. A61F 2/24

(52) U.S. Cl. CPC A61F 2/24; A61F 2/2427
USPC 623/1.26

(57) ABSTRACT

There is provided a prosthetic aortic conduit (4) comprising a replacement valve locating means (13). The replacement valve locating means can be, for example, a biocompatible radio-opaque marker (7), for example a tantalum marker. Optionally the replacement valve locating means comprises three radio-opaque markers, conveniently equi-distally spaced around the circumference of the conduit. The replacement valve locating means identifies the location of the aortic valve and facilitates later insertion of a replacement aortic valve by catheter.

The present invention relates to a vascular prosthesis, in particular a prosthesis for replacement of the aortic root.

An aneurysm of the aorta occurs when a segment of the vessel wall becomes weakened. The pressure of the blood passing through the aortic lumen causes a ballooning out of the aortic wall at the weakened segment so that the aneurysm appears as an outwards bulge in the vessel. Aortic aneurysm is a relatively common occurrence, but is a life-threatening condition since rupture of the aneurysm would cause massive internal bleeding which is usually fatal to the patient.

Thoracic aortic aneurysm (ie. an aneurysm located in the section of the aorta proximal to the heart), in addition to the potential for haemorrhage discussed above, also has potential to cause damage to the heart itself. Additionally, thoracic aortic aneurysms are also more prone to rupture.

Aortic aneurysm, including thoracic aortic aneurysm, can be successfully treated by surgery, in which the damaged vessel is replaced. Depending on the size and location of the aneurysm, surgery may require replacement of the aortic valve in addition to the neighbouring portion of aorta using a composite and valved graft onto which are reattached the two coronary arteries as originally described by Bentall and de Bono in their classical paper (Bentall H H, De Bono A: A technique for complete replacement of the ascending aorta, Thorax 1968; 23: 338-9). The “open” (Carrel button) method of coronary reimplantation was later recommended to decrease the tension on the coronary ostia while minimizing the risk of late false aneurysm formation. A modification of the standard technique was also introduced by Cabrol et al (Cabrol C et al: Complete replacement of the ascending aorta with reimplantation of the coronary arteries. New Surgical approach, J Thorac Cardiovasc Surg 1981; 81; 309-15) for those cases of difficult presentation (low lying coronary ostia, calcified coronary ostia, tissue fibrosis in redo cases) where the coronary ostia are reattached to the aortic conduit by interposition of a small conduit made in DACRON. DACRON is the Trade Name for a material formed from a straight chain polyester fibre; the material may also be known as TERYLENE.

If the aortic valve leaflets are normal, a valve-sparing aortic root remodelling procedure which keeps the natural patient valve on site is a reasonable alternative in certain individuals. David and Feindel (David T E, Feindel C M: An aortic valve-sparing operation for patients with aortic incompetence and aneurysm of the ascending aorta. J Thorac Cardiovasc Surg 1992; 103(4): 617-21) described a surgical technique where the dilated aortic root is replaced with a tube made of DACRON fibres and the native aortic valve is integrated within the graft. This method is generally known as the “Tirone David Type I aortic valve sparing procedure”. However, the lack of sinuses in a straight tube graft was found to negatively influence proper valve function, with the consequent risk of decreasing valve longevity (Kunzelman K S et al.: Aortic root and valve relationships. Impact on surgical repair J Thorac Cardiovascular Surg 1995; 109(2): 345-51).

EP 0955019 describes a modified prosthesis which mimics the sinus of Valsalva by expanding with diastolic pressure, thereby preserving valve longevity. EP 1935375 also describes a prosthesis shaped to mimic the sinus of Valsalva.

Whilst treatment of thoracic aortic aneurysm by surgery can resolve the problem of the aneurysm, subsequent failure of the aortic valve can be observed in many patients over time. Such failure is often initially observed as a reduced function of the valve and can occur at any period following surgery to resolve the aneurysm, ranging from just days after the surgery to many years later. Valve failure of this type is observed in patients irrespective of whether the aneurysm-repair surgery comprised replacement of the patient's natural aortic valve with a xenograft or mechanical valve, or whether the patient's natural valve was retained. Any xenograft or mechanical valve included in the prosthesis implant for initial aneurysm repair would inevitably have a limited lifetime.

Further surgery for replacement or repair of the failing valve is essential for patient survival, but is of increased complexity and difficulty due to the initial surgery for aneurysm repair. Consequently, the surgery for replacement of the failing valve is commonly performed by opening the thorax and requires the patient to be placed under extra-corporeal circulation. Such surgery is highly invasive and is of high risk, and requires a long recovery period. The inherent high risk to the procedure means that such surgery may not be suitable for very elderly or seriously ill patients.

An alternative to the open thoracic surgery described above involves the use of endovascular techniques, with the delivery of a heart valve being carried out by catheter. See for example U.S. Pat. No. 6,830,584. Whilst a procedure for valve delivery by catheter is far less invasive (and so is more cost-effective and facilitates better patient recovery), it is difficult to precisely locate the valve delivered by catheter at the exact position required, even using all available current imaging techniques. Whilst the existing (failing) valve can be removed and physically replaced, for simplicity any existing tissue valve can alternatively be retained in situ, with a new valve located and expanded to displace the leaflets of the existing valve.

Thus, at present, catheter delivery of a replacement heart valve cannot easily be performed where the patient has previously had a prosthetic section of thoracic aorta implanted.

There is therefore a need to be able to more confidently locate a pre-implanted prosthesis during a subsequent heart valve replacement/repair procedure conducted by endovascular procedures. The present invention seeks to address this need, by provision of a prosthesis for aneurysm repair which is adapted to accommodate any subsequent need for valve replacement or repair, in particular where the replacement valve is delivered by catheter.

The term “replacement valve” or “replacement aortic valve” as used herein refers to the valve delivered by catheter which will either replace the existing valve (if removed) or will supplant an existing tissue valve (if retained). In each case the replacement valve will operate as the heart valve, functionally replacing the previous valve. Of course, if removal of the existing valve is required, the precise location of this valve must be accurately determined to enable its removal. The presence and exact position of the previously implanted prosthesis is however difficult to visualise in a clear and unambiguous manner using fluoroscopy or any other conventional imaging system.

In each embodiment of the invention discussed below the term “comprising ” can independently be replaced with the term “consisting of”.

In one aspect, the present invention provides a prosthetic aortic conduit which comprises a replacement valve locating means. The replacement valve locating means can be one or more marker(s) positioned on the conduit to identify the location of the aortic valve. In use the marker(s) are located at or close to the end of the conduit closest to the heart (ie. the distal end of the conduit). Optionally the marker(s) identify the orientation of the valve (ie. leaflet location). In use the replacement valve locating means identifies the location and optionally the orientation of the aortic valve.

The prosthetic aortic conduit of the present invention thus comprises a distal end clearly identifiable by the presence of the replacement valve locating means and which in use is located closest to the heart, and a proximal end which in use is located away from the heart. One skilled in the art would thus be able to determine the intended orientation of the conduit prior to implantation, from the presence of the replacement valve locating means at or close to the distal end.

Optionally, the present invention provides a prosthetic aortic conduit having a distal end and a proximal end, wherein said conduit comprises at least one marker identifying the distal end of said conduit and which forms a replacement valve locating means. The marker is generally placed at or close to the distal end of the conduit to identify the position of the aortic valve following implantation into a patient. In one embodiment the marker is located from 1 cm to 5 cm from the distal end of the conduit.

Optionally, the present invention provides a prosthetic conduit having a first end and a second end with a lumen extending therethrough, said conduit comprising a replacement valve locating means comprising at least one marker defining at least one end of said conduit to identify the position of the aortic valve. In one embodiment the marker is located from 1 cm to 5 cm from the end of the conduit.

Optionally, the present invention provides a prosthetic aortic conduit having a first tubular portion and a second tubular portion connected together along a common axis and with a common lumen extending therethrough, wherein said second portion is able to deform laterally and/or is of greater internal circumference relative to the first tubular portion, and wherein said conduit comprises at least one replacement valve locating means located on said second portion. The second portion can have longitudinally extending corrugations which permit lateral expansion.

Thus, the second portion mimics the action of the sinus of Valsalva. Optionally, the conduit has a third tubular portion which is connected to the second portion along the common axis of the conduit, and with a common lumen extending therethrough. The replacement valve locating means is located on the second portion to identify the valve position. In one embodiment, the replacement valve locating means is located from 1 cm to 5 cm from the end of the second portion not connected to the first portion.

The prosthetic aortic conduit of the present invention will be implanted surgically by opening the chest cavity of the patient. The conduit will therefore not be implanted by catheter but will be manually inserted by a surgeon and fixed into position by, for example, sutures. The location and orientation of the conduit will be directly viewable by the surgeon; the markers can be accurately aligned with the commisures of the heart valve by manual manipulation of the conduit. Since the conduit of the invention is to be attached manually it will generally be non-stented.

Optionally, the prosthetic aortic conduit of the present invention can include a heart valve which replaces or supplants the patient's own (natural) heart valve at the time of implantation of the conduit. Thus, the valve can be integral with the conduit itself. An integral valve may be attached to the conduit during manufacture or (less preferably) can be attached to the conduit during surgical implantation of the conduit. Suitable heart valves can be mechanical, or can be a tissue valve or xenograft. Even where the conduit includes a heart valve, failure of such valves over time can occur so that the ability to provide a further replacement heart valve is still of significant benefit, especially since the presence of the replacement valve locating means will enable insertion of the further replacement heart valve to be conducted via a endovascular techniques, avoiding the necessity of more invasive procedures. Tissue valves or xenografts are preferred since such grafts can be retained in situ and simply supplanted by the replacement valve.

Advantageously, where the conduit comprises a heart valve, the replacement valve locating means are located to define the position of the heart valve in the conduit.

The at least one replacement valve locating means has no function in the initial surgical procedure implanting the prosthetic conduit of the present invention, but can subsequently be visualised clearly using current imaging techniques (for example by fluoroscopy) and thus enables imaging of the prosthetic conduit sufficiently to facilitate any subsequent valve replacement, in the event of valve failure or potential valve failure. The at least one replacement valve locating means provides an unambiguous and accurate indication of valve location.

Thus the replacement valve locating means define the location for precise implantation of a catheter delivered replacement valve. The replacement valve to be delivered is aligned and positioned relative to the replacement valve locating means as visualised by imaging techniques.

In the prosthetic conduit of the invention, the at least one replacement valve locating means is located either at one end of the conduit, or is located at a predetermined distance from the end of the conduit. Generally the end of the conduit having the replacement valve locating means located thereon or thereby is the distal end of the conduit ie. the end of the conduit closest to the heart. Generally the replacement valve locating means is located no further than 5 cm away from the distal end of the conduit, for example is between 2 cm to 4 cm from the distal end of the conduit.

The replacement valve locating means is generally formed of a material which is biocompatible and highly visible to at least one imaging technique. In one embodiment the replacement valve locating means comprises radio-opaque material. Suitable radio-opaque materials include gold, tungsten, platinum, tantalum or combinations thereof. In certain embodiments only portions of the replacement valve locating means need to be formed from such a material which is highly visible by an imaging technique. Tantalum is a preferred radio-opaque material for use in replacement valve locating means of the present invention, since tantalum has a low potential difference relative to nitinol and hence the possibility of galvanic corrosion is reduced in grafts containing nitinol, for example containing nitinol stents or stent elements.

The prosthetic aortic conduit of the present invention can have more than one radio-opaque marker forming the replacement valve locating means. The only limitation to the number of radio-opaque markers used, is the space available for attaching them to the conduit. Conveniently, the replacement valve locating means could comprise two, three, four, five or six radio-opaque markers. Multiple markers are preferably spaced at equal intervals around the circumference of the prosthesis.

In certain embodiments 3 markers form the replacement valve locating means, the markers being equi-distally spaced around the circumference of the prosthetic conduit.

In the conduit of the present invention the replacement valve locating means can comprise 3 equidistant markers, spaced around the circumference of the conduit. In this embodiment, the replacement valve locating means can comprise markers located to be aligned with the commisures of a heart valve during implantation of the prosthesis. The markers can therefore be aligned with the commisures of a replacement heart valve, delivered by catheter. This ability to rotationally align a replacement heart valve ensures that no coronary artery will be obstructed by the new replacement valve, as the replacement heart valve will have the same rotational orientation of the valve it supplants.

In one embodiment, where three or more markers are present in the replacement valve locating means, one of the markers is of a different unique shape or dimension from the other markers and so can be individually identified by imaging. This unique marker is preferably located to enable identification of the non-coronary sinus of the heart valve. The replacement valve locating means can comprise markers sized and shaped to facilitate their attachment to the prosthesis. In one embodiment, the markers are attached to the prosthesis by suturing with a sewing needle and thread (eg. suture). The markers can be attached using equi-spaced stitches. Conveniently 3, 4, 5, or 6 equi-spaced stitches are used. Alternatively, the markers can be integrally formed within the conduit itself, ie. are woven or knitted into position or are located between two layers of material forming the prosthetic conduit.

Each or any of the marker(s) can have a ring or “doughnut” shape.

Alternatively, each or any of the marker(s) can have a button-like shape, that is a plate (of any shape including round, oval or rectangular) having apertures therethrough to permit passage of a needle and thread.

Other suitable shapes for each or any of the marker(s) include dumbbell, figures of 8, sections of wire and the like.

Other shapes of marker are also possible. Any suitable shape which enables easy attachment to the graft (for example by sewing) and provides good visibility and distinctiveness (ie. can be distinguished from simple bright spots) is suitable.

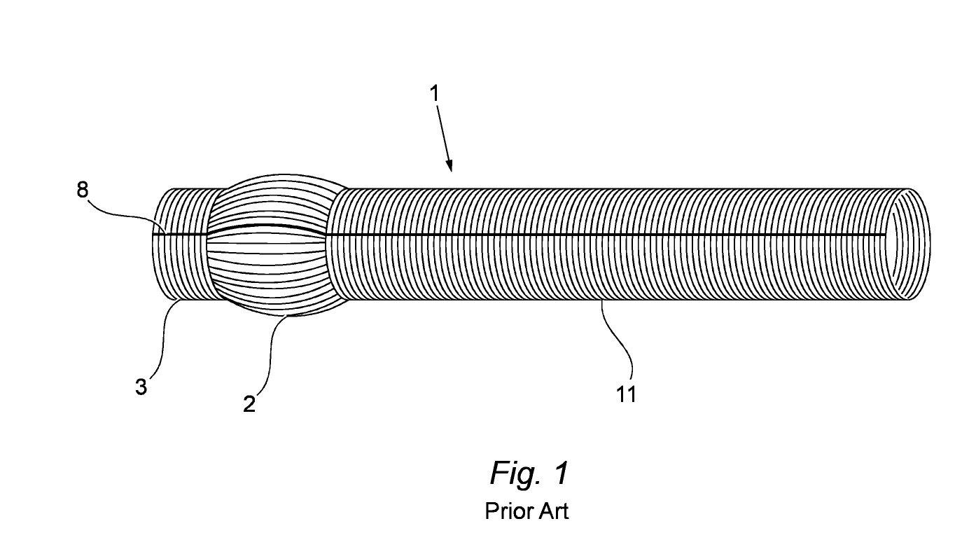

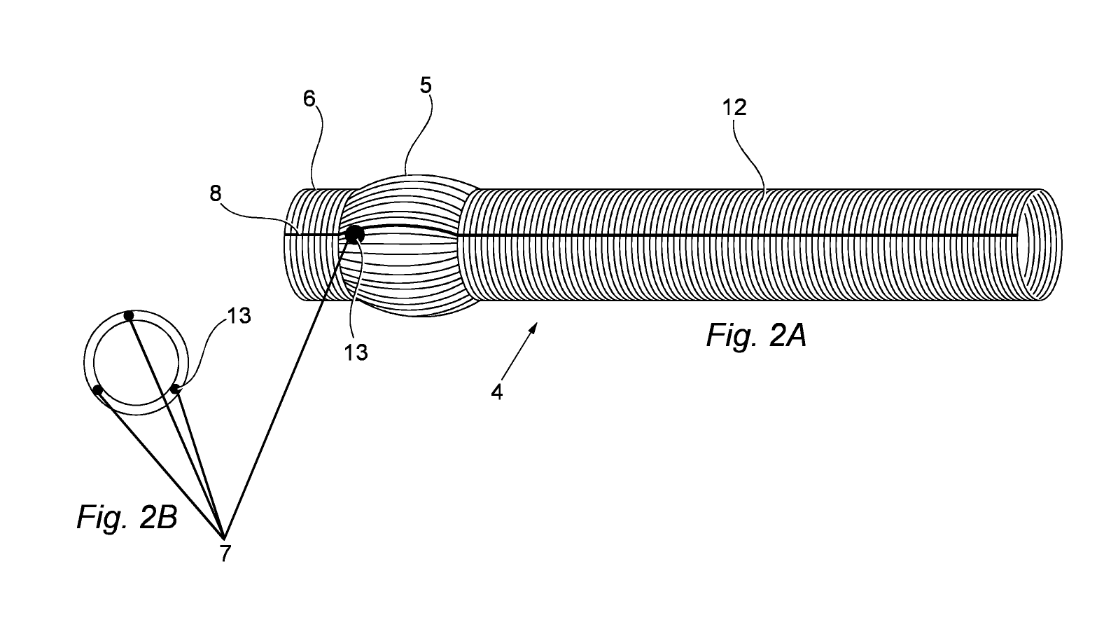

It is advantageous for the markers of the replacement valve locating means to be radio-opaque so that the prosthesis position is determinable using fluoroscopy. Careful selection of marker location enables the angle of tilt of the prosthetic conduit to be clearly established using such imaging procedures. For example, 3 radio-opaque markers can be used in the replacement valve locating means, each of which are placed equi-distantly around the distal end of the conduit (ie. the end closest to the heart valve). Where the prosthesis comprises a mock sinus of Valsalva portion (see, for example reference 2 of FIG. 1 or reference 5 of FIG. 2A), the replacement valve locating means can be placed at the distal end of this portion or from 1 to 5 cm away from the distal end of this portion.

The prosthetic conduit can be formed of any suitable biocompatible material. Mention can be made of polyester, polypropylene, polyethylene and PTFE, including ePTFE, as suitable materials. A preferred material is DACRON. DYNEEMA (a polyethylene) is also suitable. The material used to form the conduit can optionally be coated. Suitable prostheses are described in EP 0955019A, EP 1935375 and WO 2006/038031. Mention can be made of GELWEAVE, a woven polyester material coated with gelatine as a suitable material for the conduit. An elastomer sealed polyester graft is particularly useful where the conduit includes an integral tissue heart valve for implantation since wet storage of the prosthesis is essential to maintain the tissue valve.

Optionally, the prosthesis comprises a first tubular portion, a second tubular portion and a third tubular portion connected together along a common axis and has a common lumen extending therethrough, wherein said second tubular portion is laterally deformable and/or is of greater cross-sectional area relative to the first or third portion, wherein said prosthesis further comprises a heart valve comprising three commisures, wherein said valve is located at the junction between the second and third tubular portions, and wherein the prosthesis comprises a replacement valve locating means having three markers, wherein each marker is attached to the second or third tubular portion to identify the location of a heart valve commisure.

In the above embodiment, the markers of the replacement valve locating means are preferably formed from tantalum. Further, in the above embodiment, the markers are preferably ring-shaped or button-like, with optionally one marker being of a unique shape or size and aligned to identify the non-coronary sinus of the heart valve.

The prosthesis of the invention will be implanted by surgical opening of the patient's chest. Accordingly, the markers have no function during the implantation of the prosthesis since the location and orientation of the prosthesis will be directly viewable by the surgeon.

In a further aspect, the present invention provides a method of treatment of a patient in need thereof, said method comprising:
a) replacing a portion of the thoracic aorta of a patient with a prosthesis having a replacement valve locating means.

The step of replacing a portion of the thoracic aorta will involve surgical implantation of the prosthesis by surgical opening of the chest wall of the patient.

Optionally the method can further comprise:
b) subsequently using said replacement valve locating means to identify the prosthesis by imaging; and
c) delivering an aortic heart valve to the patient by catheterisation techniques.

Delivery of the aortic heart valve is to a location within the prosthesis identified by the replacement valve locating means.

In a further aspect, the present invention provides a method of treatment of a patient in need thereof, said method comprising:
a) replacing a portion of the thoracic aorta of the patient with a prosthetic conduit having a distal end and a proximal end, and a lumen extending therethrough, wherein said conduit has a replacement valve locating means located at or close to the distal end of said conduit to define the position of a first aortic heart valve.

Optionally the method can further comprise:
b) subsequently identifying the location of said replacement valve locating means;
c) determining the location of said first aortic heart valve from the identified location of said replacement valve locating means;
d) optionally using said determined location of said first aortic heart valve to remove said first aortic valve by catheter; and
e) using said identified location of said replacement valve locating means to locate a second aortic heart valve, wherein said second aortic heart valve is delivered by catheter.

Where step d) is present and the first aortic heart valve is removed, the second aortic heart valve replaces that valve. The first aortic heart valve can then be the patient's own natural valve, can be a xenograft (tissue) valve or can be a mechanical valve. However step d) is optional and it can be more convenient to allow the first aortic heart valve to remain in situ, despite any reduction in function. In this embodiment the second aortic heart valve supplants the first aortic valve heart and takes over its function. Optionally, the second aortic heart valve is simply expanded to displace the leaflets of the first aortic heart valve. Generally the first aortic heart valve will be a tissue valve if it is allowed to remain in situ. The first aortic heart valve could be the patient's own (natural) heart valve or could be a xenograft tissue valve.

Optionally, in the above method, the replacement valve locating means has three markers equi-spaced around the circumference of the conduit lumen.

Optionally, in any of the above methods, the first aortic heart valve is part of the prosthetic conduit, and the replacement valve locating means has three markers equi-spaced around the circumference of the conduit lumen to identify the location of the commisures of the first aortic heart valve. Thus, in step e) the second aortic heart valve is rotationally positioned such that the commisures of the second aortic heart valve are aligned with the markers.

The present invention also provides a method of treatment of a patient in need thereof, wherein said method comprises:
a) replacing a portion of the thoracic aorta of the patient with a prosthesis comprising first, second and third tubular portions connected together along a common axis and having a common lumen extending therethrough, wherein said second portion is laterally deformable and/or has a lumen of greater cross-sectional area than either the first or third tubular portions, wherein said prosthesis comprises a first heart valve and a replacement valve locating means to identify the position said first heart valve.

Optionally the method further comprises:
b) subsequently identifying the location of said replacement valve locating means;
c) determining the location of said first heart valve from the identified location of said replacement valve locating means;
d) optionally using said determined location of said first heart valve to remove said first heart valve by catheter; and
e) using said identified location of said replacement valve locating means to locate a second aortic heart valve, wherein said second aortic heart valve is delivered by catheter.

Where step d) is present and the first heart valve is removed, the second aortic heart valve replaces the first heart valve. The first aortic heart valve can then be the patient's own natural valve, can be a xenograft (tissue) valve or can be a mechanical valve. However step d) is optional and it can be more convenient to allow the first heart valve to remain in situ, despite any reduction in function. In this embodiment the second aortic heart valve supplants the first valve heart and takes over its function. Optionally, the second aortic heart valve is simply expanded to displace the leaflets of the first heart valve. Generally the first aortic heart valve will be a tissue valve if it is allowed to remain in situ. The first aortic heart valve could be the patient's own (natural) heart valve or could be a xenograft tissue valve.

In the above method, the replacement valve locating means can have three markers each of which identify a commisure of the first heart valve integral to said prosthesis. Optionally one of the markers has a unique shape or size and is positioned on the prosthesis to identify the non-coronary sinus of the first heart valve. Optionally the replacement valve locating means are used to rotationally align the second aortic valve to the same approximate orientation of the first heart valve so that the coronary arteries are not obstructed.

In a further aspect, the present invention provides a method of replacing an aortic heart valve in a patient who has previously had surgery to insert an aortic conduit comprising replacement valve locating means, said method comprising:
a) subsequently using said replacement valve locating means to identify the prosthesis by imaging; and
b) delivering an aortic heart valve to the patient by catheterisation techniques.

The aortic conduit used in this method is a conduit according to the present invention having any or all of the features as described above.

In a further aspect, the present invention provides a method of determining the location of a heart valve in a patient, wherein said patient has had a prosthetic aortic conduit according to the invention surgically implanted, said method comprising:
a) imaging the patient's chest area using a technique which detects the replacement valve locating means; and
b) determining the location of the heart valve from the detected replacement valve locating means.

Fluoroscopy can be used as the imaging technique.

Optionally, in this aspect, the prosthesis implanted into the patient has three or more markers (preferably equidistantly spaced around the circumference of the prosthesis) so that the orientation of the heart valve can be determined in addition to its location.

Where the prosthesis implanted into the patient includes three or more equally spaced markers, with one of the markers having a uniquely identifiable shape or size, the method further includes the step of identifying the non-coronary sinus of the heart valve.

The prosthesis referenced in the above method can have any or all of the features described above.

Preferred or alternative features of each aspect or embodiment of the invention apply mutatis mutandis to each other aspect or embodiment of the invention, unless the context demands otherwise.

The present invention will now be further described by reference to the following figures, in which:

FIG. 1: is a schematic illustration of a prior art prosthetic conduit suitable for aortic aneurysm repair;

FIG. 2A: is a schematic illustration of a prosthetic conduit according to the present invention;

FIG. 2B: is a cross section of the conduit of FIG. 2A showing the spacing of the markers;

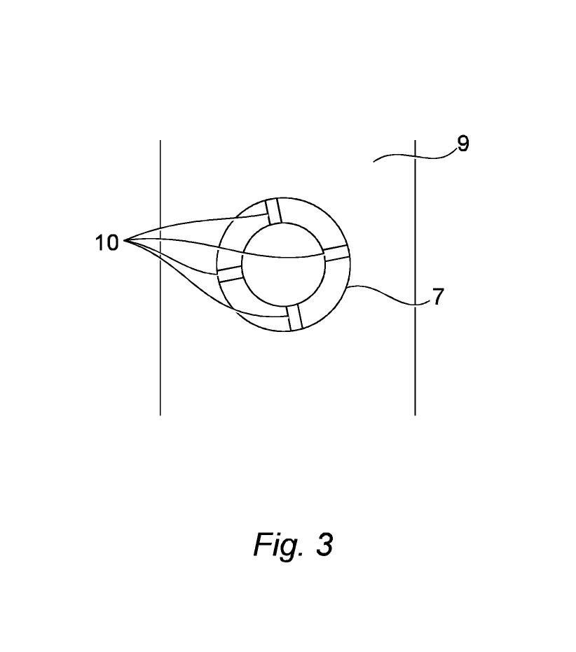

FIG. 3: is a plan view of a marker used in the prosthetic conduit according to the present invention and illustrated in FIG. 2A.

Referring to the drawings, FIG. 1 shows an aortic conduit (1) as used in the prior art and described in detail in EP 0955019. As illustrated, the conduit (1) comprises a first tubular portion (11), a second tubular portion (2) and a third tubular portion (3). Each of the tubular portions are connected along a substantially common axis and have a common lumen extending along the whole prosthesis (1). The first portion (11) is attached to the second portion (2) at one end of the second portion. The third portion (3) is attached to the other end of the second portion (2). As shown, the prior art prosthesis (1) includes a coloured thread (8) which extends along the outside of the graft and provides visual indication of any twisting of distortion of the graft. The first portion (11) includes circumferentially extending corrugations which allows the first portion (11) to deform in the longitudinal direction. The second portion (2) includes longitudinally extending corrugations which allow the second portion (2) to be expandable in a lateral direction. The deformation of the second portion (2) in a lateral direction enables this portion of the prosthetic conduit (1) to mimic the sinuses of Valsalva. The third portion (3) is similar to the first portion (11) in that it comprises annular corrugations successively provided along the longitudinal axis of the conduit (1) and so is able to expand in a longitudinal direction. Suitable materials for the first, second and third tubular portions include DACRON or PTFE material. The prior art prosthetic conduit illustrated in FIG. 1 includes no replacement valve locating means which can be visualised using imaging techniques. As a result, the synthetic conduit (1) will be highly difficult to visualise following its implantation into a patient. A xenograft (eg. porcine) valve or the patient's own aortic heart would usually be incorporated into the prosthesis (1). The valve (not shown) would be located in the third portion (3) adjacent its junction with the second portion (2).

FIG. 2A illustrates an exemplary prosthetic conduit (4) of the present invention. The prosthetic conduit (4) comprises a first tubular portion (12), a second tubular portion (5) and a third tubular portion (6) which are substantially identical to the first, second and third tubular portions (11, 2, 3) of the prior art graft (1) illustrated in FIG. 1. In particular, the first and third tubular portions (12, 6) include annular corrugations provided successively along the longitudinal axis of each portion and which provide a degree of expansion in the longitudinal direction, enabling the conduit (4) to increase its length. The second portion (5) has longitudinally orientated corrugations which enable easy expansion of this portion of the graft in the lateral direction. Thus, the second portion (5) will act, upon implantation, as a “sinotubular junction” and its internal diameter can vary during the cardiac cycle (systole/diastole) as in the natural aortic root. Thus, the second portion (5), when filled with blood under pressure, will stretch in the direction transverse to the longitudinal axis of the prosthesis (the lateral direction) mimicking the sinuses of Valsalva. The prosthetic conduit can conveniently be formed from PTFE or polyester. Woven polyester coated with a sealant such as gelatin is particularly advantageous, especially where the conduit includes an integral xenograft heart valve. Also shown in FIG. 2A is a coloured thread (8) extending longitudinally along the whole length of the conduit (4). This coloured thread provides a visual indication of any twisting or other distortion of the graft (4) during its implantation.

The prosthetic conduit (4) also includes a replacement valve locating means (13) formed from three radio-opaque markers (7). Only one marker (7) can be seen in the view presented in FIG. 2A, since these markers are equally spaced around the circumference of the conduit (4) as illustrated in FIG. 2B. The replacement valve locating means (13) is located at the distal end of the prosthetic conduit (4) and just before the heart valve (either a xenograft heart valve or the patient's own valve inserted into the prosthesis). As shown, the replacement valve locating means (13) is located at the distal end of the second portion (5). The markers (7) can suitably be formed of tantalum.

In use, the surgeon will open the patient's chest cavity and insert the prosthesis (4), replacing the diseased or damaged portion of the patient's aorta. Where the patient's own heart valve is retained, the surgeon will orientate the prosthesis so that the markers (7) align with the commisures of the heart valve, in order to identify the valve location and orientation. Similarly, should a xenograft heart valve be utilised, the surgeon will attach the xenograft valve to the prosthesis so that the markers (7) are aligned with the commisures of that valve. The prosthesis will then be fixed into location by suitable means such as sutures and the patient's chest wall will be closed. Since the patient's chest cavity has been surgically opened, the prosthesis is located and orientated manually by the surgeon. The markers are not visualised by imaging techniques during insertion of the prosthesis since the surgeon inserts the graft manually and can view the prosthesis directly. Should the heart valve require replacement at any future point (possibly years later), the heart valve can be clearly visualised using imaging such as fluoroscopy by reference to the marker(s) allowing endovascular placement of a replacement valve.

FIG. 3 shows the marker (7) of the replacement valve locating means (13) in FIG. 2A, 2B in detail. As is illustrated, marker (7) is a doughnut-shape formed from a highly radio-opaque material, such as tantalum. Markers (7) are attached to the outer surface (9) of the second portion (5) of the conduit (4) by sutures (10). Four sutures (10) are shown equi-spaced around the marker (7) attaching it to the outer surface (9) of second portion (5).

In use, the replacement valve locating means (13) is located at a pre-determined position on the conduit (4) relative to the heart valve. Conveniently, the markers (7) of the replacement valve locating means (13) are aligned or otherwise located at a predetermined position relative to the commisures of the heart valve in the prosthesis or of the patient's own heart valve. The markers (7) are easily visualised by imaging techniques such as fluoroscopy. In the event of failure or reduced function of the heart valve, visualisation of the markers (7) of the replacement valve locating means (13) can be used to accurately determine the position of the heart valve within the conduit (4), so that it can be accurately be removed using endovascular techniques without damage to the walls of the conduit (4). Valve removal is, however, optional. More importantly, a replacement heart valve can be delivered to the position defined by replacement valve locating means (13) and rotationally aligned to avoid obstruction of the coronary arteries and, if the existing valve is retained, expanded to displace the leaflets of the existing valve.

All documents referred to in this specification are herein incorporated by reference. Various modifications and variations to the described embodiments of the inventions will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes of carrying out the invention which are obvious to those skilled in the art are intended to be covered by the present invention.

1. A prosthetic aortic conduit comprising a replacement valve locating means configured to identify a position of an aortic valve.

2. The prosthetic aortic conduit as claimed in claim 1 wherein said replacement valve locating means comprises at least one radio-opaque marker.

3. The prosthetic aortic conduit as claimed in claim 1 wherein said replacement valve locating means comprises at least three radio-opaque markers.

4. The prosthetic aortic conduit as claimed in claim 3 wherein said at least three radio-opaque markers are spaced at equal intervals around a circumference of the prosthetic aortic conduit.

5. The prosthetic aortic conduit as claimed in claim 4 wherein one of said at least three radio-opaque markers is uniquely identifiable.

6. The prosthetic aortic conduit as claimed in claim 3 wherein said at least three radio-opaque markers are located on the prosthetic aortic conduit for alignment with commisures of the aortic valve.

7. The prosthetic aortic conduit as claimed in claim 5 wherein said uniquely identifiable marker is located on the prosthetic aortic conduit for alignment with a non-coronary sinus of the aortic valve.

8. The prosthetic aortic conduit as claimed in claim 1 for non-endovascular insertion.

9. The prosthetic aortic conduit as claimed in claim 1, comprising an aortic valve.

10. The prosthetic aortic conduit as claimed in claim 1, comprising a mock sinus of Valsalva.

11. The prosthetic aortic conduit as claimed in claim 10 comprising:
a first tubular portion, a second tubular portion and a third tubular portion connected together along a common axis;
a common lumen extending through the first tubular portion, the second tubular portion, and the third tubular portion;
wherein said second tubular portion is laterally deformable; and
wherein said replacement valve locating means is located on said second portion.

12. The prosthetic aortic conduit as claimed in claim 10 comprising:
a first tubular portion, a second tubular portion and a third tubular portion connected together along a common axis;
a common lumen extending through the first tubular portion, the second tubular portion, and the third tubular portion;
wherein said second tubular portion is laterally deformable;
wherein said prosthetic aortic conduit further comprises a heart valve comprising three commisures;
wherein said valve is located at a junction between the second and third tubular portions; and
wherein the prosthetic aortic conduit comprises a replacement valve locating means having three markers, wherein each marker is attached to the second or third tubular portion to identify the location of a heart valve commisure.

13. A method of determining a location of a heart valve in a patient, wherein said patient has had a prosthetic aortic conduit as claimed in claim 1 surgically implanted, said method comprising:
imaging the patient's chest area using a technique able to detect the replacement valve locating means; and
determining the location of the heart valve from the detected replacement valve locating means.

14. The method of claim 13 wherein an orientation of said heart valve is determined.

15. A method of treatment of a patient in need thereof, said method comprising:
replacing a portion of a thoracic aorta of the patient with a prosthesis having a replacement valve locating means configured to identify an aortic valve.

16. The method of claim 15 comprising:
subsequently using said replacement valve locating means to identify the prosthesis by imaging; and
delivering a replacement aortic heart valve to the patient by catheterisation techniques.

17. A method of treating a patient in need thereof, said method comprising:
replacing a portion of a thoracic aorta of the patient with a prosthetic conduit having a distal end and a proximal end, and a lumen extending therethrough; and
wherein said conduit has a replacement valve locating means located at or close to the distal end of said conduit to define a position of a first aortic heart valve.

18. The method of claim 17 comprising:
subsequently identifying a location of said replacement valve locating means;
determining a location of said first aortic heart valve from the identified location of said replacement valve locating means;
optionally using said determined location of said first aortic heart valve to remove said first aortic valve by catheter; and
using said identified location of said replacement valve locating means to locate a second aortic heart valve, wherein said second aortic heart valve is delivered by catheter.

19. A method of treatment of a patient in need thereof, said method comprises:
replacing a portion of a thoracic aorta of the patient with a prosthesis comprising first, second and third tubular portions connected together along a common axis and having a common lumen extending therethrough;
wherein said second portion is laterally deformable; and
wherein said prosthesis comprises a first heart valve and a replacement valve locating means to identify the position said first heart valve.

20. The method of claim 19, comprising:
subsequently identifying a location of said replacement valve locating means;
determining a location of said first heart valve from the identified location of said replacement valve locating means;
optionally using said determined location of said first heart valve to remove said first heart valve by catheter; and
using said identified location of said replacement valve locating means to locate a second aortic heart valve, wherein said second aortic heart valve is delivered by catheter.

21. A method of replacing an aortic heart valve in patient who has previously had surgery implanting an aortic conduit according to claim 1, said method comprising:
subsequently using said replacement valve locating means to identify a prosthesis by imaging; and
delivering an aortic heart valve to the patient by catheterisation techniques.

22. The method of claim 13 wherein said replacement valve locating means comprises at least three radio-opaque markers.

23. The method as claimed in claim 22 wherein said radio-opaque markers are spaced at equal intervals around a circumference of the prosthetic aortic conduit.

24. The method of claim 23 wherein said marker is aligned with commissures of the aortic valve.

25. The method of claim 24 wherein one of said markers is uniquely identifiable and is aligned with a non-coronary sinus of the aortic valve.
